(19) United States
(12) Patent Application Publication
(10) Pub. No.: US 2009/0062231 A1
(43) Pub. Date: Mar. 5, 2009

(54) Appetite Suppressant Composition and Method of Appetite Suppression

(76) Inventors: Ann Marie O'Mara, Limerick (IE); Brendan Joseph O'Mara, Clare (IE)

Correspondence Address: AMIN HALLIHAN, LLC, 444 NORTH ORLEANS STREET, SUITE 400, CHICAGO, IL, 60654, US

(21) Appl. No.: 11/848,250

(22) Filed: Aug. 31, 2007

(51) Int. Cl. A61K 31/715; A61P 3/04

(52) U.S. Cl. 514/54

(57) ABSTRACT

The present invention provides an appetite suppressant composition including alginate, hydroxycitric acid, and piperine. The present invention also provides an appetite suppressant composition including alginate, hydroxycitric acid, and garcinol. The present invention further provides a method of suppressing appetite utilizing an appetite suppressant composition for creating an immediate and sustained feeling of satiety; reducing total cholesterol, glucose, insulin, and triglyceride levels; and preventing gastric reflux.

### FIELD OF THE INVENTION

The present invention relates to a composition containing alginate used for suppressing the appetite of humans. The present invention further relates to a composition containing alginate used to reduce the body mass index, food digestion and absorption, and cholesterol, blood glucose and insulin levels in humans. The present invention is designed to be ingested and can be taken as a beverage.

### BACKGROUND OF THE INVENTION

Weight loss and dieting are constant struggles for a lot of people. Appetite suppression is one method of controlling food intake and ultimately losing weight. Another method of losing weight is changing diet and/or exercise habits, which is often difficult to do, especially in the fast-paced world where it is difficult to find time to exercise or make a healthy meal. In order to lose weight, calorie intake must be reduced. Appetite suppression is one way to eliminate cravings, control food intake and reduce calorie intake. Generally currently used appetite suppressants include systemic drugs which have many side effects such as dependency, resistance, insomnia, drowsiness, irritability or depression. Accordingly, there is a demand for an appetite suppressant composition which is safe, effective, and without harmful side effects.

### SUMMARY OF THE INVENTION

One object of the present invention is for an ingested appetite suppressant composition having components with synergistic effects.

A more specific object of the present invention is to overcome one or more of the problems discussed above.

Another object of the present invention is for an appetite suppressant composition having the ability to sustain and immediately give a feeling of satiety.

Still another object of the present invention is for an appetite suppressant composition having the ability to reduce body mass index, and cholesterol, triglycerides, glucose and insulin levels.

A further object of the present invention is for an appetite suppressant composition which increases the bioavailability of the appetite suppressant composition components.

A still further object of the present invention is for an appetite suppressant composition which impacts the GLP-1 and ghrelin hormones which are involved in the regulation of hunger and satiety.

An even further object of the present invention is for an appetite suppressant composition which provides soluble fiber without bloating.

In accordance with the present invention, a composition containing alginate for suppressing the appetite has been developed. In one form, the appetite suppressant composition can be in the form of a beverage, capsule, pill, lozenge, or other suitable form.

The appetite suppressant composition in one form of the invention includes alginate, hydroxycitric acid, and garcinol.

The appetite suppressant composition in another form of the invention includes alginate, hydroxycitric acid, and piperine.

In one embodiment, the appetite suppressant composition can include and/or consist of about at least about 0.1% (w/v) alginate, and at least about 0.18% (w/v) of a composition including hydroxycitric acid and garcinol, in 500 mL water.

In another embodiment, the appetite suppressant composition can include and/or consist of at least about 0.1% (w/v) alginate, at least about 0.001% (w/v) piperine, and at least about 0.18% (w/v) hydroxycitric acid, in 500 mL water.

One form of the invention also relates to a method of suppressing appetite including administering the appetite suppressant composition of the present invention.

Another form of the invention relates to a method of reducing total cholesterol, glucose, insulin and triglyceride levels including administering the appetite suppressant composition of the present invention.

Another form of the invention relates to a method of creating a feeling of satiety including administering the appetite suppressant of the present invention.

These and other embodiments are more fully described in connection with the detailed description.

### DETAILED DESCRIPTION OF THE INVENTION

The present invention relates to a composition containing alginate used for suppressing the appetite of humans. One form of the invention further relates to a composition used to reduce the body mass index, and cholesterol, blood glucose and insulin levels. One form of the composition is designed to be ingested and can be taken as a beverage.

The term “alginate” will be defined to include alginate, alginic acid and its various forms, and other salts of alginic acid.

The term “hydroxycitric acid” will be defined to include hydroxycitric acid, an active salt of hydroxycitric acid or a combination thereof.

One embodiment of the present invention relates to an appetite suppressant composition having alginate; hydroxycitric acid; and a hydroxycitric acid bioavailability increasing component, such as piperine and/or garcinol.

One embodiment of the present invention relates to an appetite suppressant composition having alginate; hydroxycitric acid; and garcinol.

A further embodiment of the present invention relates to an appetite suppressant composition having alginate; and a composition including hydroxycitric acid and garcinol. The composition including hydroxycitric acid and garcinol can be GARCITRIN®, a standardized extract manufactured by the Sabinsa Corporation, containing about 50% hydroxycitric acid and about 5% garcinol. GARCITRIN® can contain synthetic or natural hydroxycitric acid from any Garcinia species.

Another embodiment of the present invention relates to an appetite suppressant composition having alginate; hydroxycitric acid; and piperine.

The synergistic effects of the components of the appetite suppressant composition in one form are believed to immediately produce and sustain a feeling of satiety. The appetite suppressant composition in one form is also believed to reduce body mass index, and cholesterol, triglycerides, blood glucose and insulin levels. The appetite suppressant composition further is believed to impact the GLP-1 and ghrelin hormones which are involved in regulating hunger and satiety. The appetite suppressant composition also further does not cause bloating from the soluble fiber ingested from the alginate.

Alginate is a component of the cell walls of brown seaweed species. Alginate is a salt of alginic acid, a linear polymer containing mannuronic and glucuronic acids. When alginate comes in contact with hydrogen ions and divalent cations, such as calcium, its polymer strands are cross-linked and it forms an irreversible gel. Alginate can also form a gel in the presence of hydrogen ions, whereby it becomes insoluble and forms a reversible gel. Gels formed by alginate have been used as cosmetic masks, and in combination with hydroxycitric acid, have been used for the external reduction of excess fat on the face and body. Alginate has also been used as a food stabilizer. Further, the intake of dietary alginates alone can result in a number of potentially beneficial physiological effects, such as prevention of acid reflux when used in combination with sodium bicarbonate.

When ingested, alginate forms a gel in the stomach which is believed to produce an immediate and sustained effect of satiety. Additionally, alginate is believed to block the absorption of metal ions, cholesterol and glucose by binding to them, wherein the levels of total cholesterol, blood glucose, and insulin can be reduced.

Suitably, the alginate of the present invention can have a concentration of glucuronic acid residues that is greater than a concentration of mannuronic acid residues to provide improved alginate gel formation. Suitably, the alginate of the present invention can have a concentration of mannuronic acid that is less than a concentration of glucuronic acid residues to provide improved alginate gel formation. Further, alginate is believed to inhibit protease activity resulting in decreased glycemic load from protein containing meals.

In one embodiment of the present invention, the appetite suppressant composition can include about 0.01% to 10% (w/v) alginate. In a further embodiment of the present invention, the appetite suppressant composition can include about 0.05% to 6% (w/v) alginate. The appetite suppressant composition can still further include about 0.1% to 1.5% (w/v) alginate. In an even further embodiment of the present invention, the appetite suppressant composition can include at least about 0.1% (w/v) alginate.

In another embodiment of the present invention, the appetite suppressant composition can include about 0.05 g to 50 g alginate in 500 mL of water. In another embodiment of the present invention, the appetite suppressant can include about 0.25 g to 30 g alginate in 500 mL of water. The appetite suppressant composition can still further include about 0.5 g to 7.5 g alginate in 500 mL of water. In an even further embodiment of the present invention, the appetite suppressant composition can include at least about 0.5 g alginate in 500 mL of water.

In a further embodiment of the present invention, the appetite suppressant composition can include at least about 0.5 g alginate.

Hydroxycitric acid enters the bloodstream when ingested. Hydroxycitric acid alone has been shown to inhibit the ATP-citrate lyase enzymes responsible for converting carbohydrates into fats. This inhibition causes the production of glycogen in the liver which produces a satiety signal. Thus, hydroxycitric acid is believed to reduce the amount of fat synthesis which can improve the blood lipid profiles wherein the levels of cholesterol and triglycerides in the blood are reduced. The hydroxycitric acid is also believed to prevent gas production and bloating from the ingestion of alginate, a source of soluble fiber.

Suitably, the hydroxycitric acid of the present invention can be in the form of an active salt such as sodium hydroxycitric acid, calcium hydroxycitric acid and/or potassium hydroxycitric acid wherein the hydrogen ions do not cause the alginate to form a gel before the appetite suppressant composition enters the stomach. In one embodiment, the appetite suppressant composition can include sodium and/or potassium hydroxycitric acid due to its ability to maintain a low viscosity of the alginate, while allowing the alginate to form a gel in the presence of divalent cations and hydrogen ions.

In one embodiment of the present invention, the appetite suppressant composition can include about 0.0001% to 1% (w/v) hydroxycitric acid. In a further embodiment of the present invention, the appetite suppressant composition can include about 0.04% to 0.4% (w/v) hydroxycitric acid. The appetite suppressant composition can still further include about 0.1% to 0.2% (w/v) hydroxycitric acid. In an even further embodiment of the present invention, the appetite suppressant composition can include about at least 0.18% (w/v) hydroxycitric acid.

In another embodiment of the present invention, the appetite suppressant composition can include about 50 mg to 5 g hydroxycitric acid in 500 mL of water. In another embodiment of the present invention, the appetite suppressant can include about 200 mg to 2 g hydroxycitric acid in 500 mL of water. The appetite suppressant composition can still further include about 500 mg to 1 g hydroxycitric acid in 500 mL of water. In an even further embodiment of the present invention, the appetite suppressant composition can include at least about 900 mg hydroxycitric acid in 500 mL of water.

In a further embodiment of the present invention, the appetite suppressant composition can include at least about 900 mg hydroxycitric acid.

Garcinol is a polyisoprenylated benzophone isolated from Garcinia cambogia and Garcinia indica. The rind of Garcinia cambogia contains an isomer of hydroxycitric acid. Garcinol is known to be an anti-oxidant, anti-carcinogen, and anti-microbial.

During the alginate gel formation in the stomach, a portion of the hydroxycitric acid can be trapped by the alginate gel and may not be available for absorption. The garcinol in one form of the invention can increase the bioavailability of the portion of the hydroxycitric acid not trapped during the alginate gel formation wherein the absorption of the hydroxycitric acid which is an appetite suppressant can increase. Thus the garcinol can negate the loss of the hydroxycitric acid trapped in the alginate gel formation. Without the garcinol it can take up to ninety minutes to feel satiety, but with the garcinol there is believed to be an immediate feeling of satiety resulting from the increased absorption of the hydroxycitric acid.

In one embodiment of the present invention, the appetite suppressant composition can include at least about 25 mg garcinol.

In another embodiment of the present invention, the appetite suppressant composition can include about 0.0001% to 1% (w/v) of the composition including hydroxycitric acid and garcinol. In a further embodiment of the present invention, the appetite suppressant composition can include about 0.04% to 0.4% (w/v) of the composition including hydroxycitric acid and garcinol. The appetite suppressant composition can still further include about 0.1% to 0.2% (w/v) of the composition including hydroxycitric acid and garcinol. In an even further embodiment of the present invention, the appetite suppressant composition can include about at least 0.18% (w/v) of the composition including hydroxycitric acid and garcinol.

In another embodiment of the present invention, the appetite suppressant composition can include about 50 mg to 5 g of the composition including hydroxycitric acid and garcinol in 500 mL of water. In another embodiment of the present invention, the appetite suppressant can include about 200 mg to 2 g of the composition including hydroxycitric acid and garcinol in 500 mL of water. The appetite suppressant composition can still further include about 500 mg to 1 g of the composition including hydroxycitric acid and garcinol in 500 mL of water. In an even further embodiment of the present invention, the appetite suppressant composition can include at least about 900 mg of the composition including hydroxycitric acid and garcinol in 500 mL of water.

In a further embodiment of the present invention, the appetite suppressant composition can include at least about 900 mg of the composition including hydroxycitric acid and garcinol.

The piperine in another form of the invention can increase the bioavailability of the portion of hydroxycitric acid not trapped during the alginate gel formation wherein the absorption of the hydroxycitric acid which is an appetite suppressant can increase. Thus the piperine can negate the loss of the hydroxycitric acid trapped in the alginate gel formation. Without the piperine it can take up to ninety minutes to feel satiety, but with the piperine there is believed to be an immediate feeling of satiety resulting from the increased absorption of the hydroxycitric acid.

Piperine does not generally dissolve in water. Suitably, the piperine of the present invention can be bound to dextrin wherein the piperine/dextrin complex dissolves in water when the appetite suppressant composition is taken as a beverage.

In one embodiment of the present invention, the appetite suppressant composition can include about 0.0002% to 1% (w/v) piperine. In a further embodiment of the present invention, the appetite suppressant composition can include about 0.0006% to 0.4% (w/v) piperine. The appetite suppressant composition can still further include about 0.001% to 0.006% (w/v) piperine. In an even further embodiment of the present invention, the appetite suppressant composition can include about at least 0.001% (w/v) piperine.

In another embodiment of the present invention, the appetite suppressant composition can include about 1 mg to 5 g piperine in 500 mL of water. In another embodiment of the present invention, the appetite suppressant can include about 3 mg to 2 g piperine in 500 mL of water. The appetite suppressant composition can still further include about 5 mg to 30 mg piperine in 500 mL of water. In an even further embodiment of the present invention, the appetite suppressant composition can include about at least 5 mg piperine in 500 mL of water.

In a further embodiment of the present invention, the appetite suppressant composition can include at least about 5 mg piperine.

In another embodiment, the appetite suppressant composition can include a flavor component, a sweetener component and/or combinations thereof for improved taste.

In one embodiment of the present invention, the appetite suppressant composition can be a beverage. In one form, the beverage is a clear, non-viscous beverage. In another form, the appetite suppressant composition is dissolved in water to form a beverage. Suitably, the appetite suppressant composition can have a flavor and can be consumed as a snack. In another embodiment, the appetite suppressant composition can include 1 to 3 drops of fruit concentrate. In another embodiment of the invention, the appetite suppressant composition can be in the form of a pill, a capsule, a tablet, a lozenge, a powder, a chewable, or syrup.

Alginate is not very water soluble and may not dissolve in water because it binds to the divalent cations and forms a gel, especially when the water is rich in minerals such as calcium and magnesium. Agitation, dispersion and hydration can be used to dissolve the alginate in water. Suitably, the water of the present invention can be deionized or low in minerals wherein the alginate dissolves and only forms a gel when the appetite suppressant composition enters the stomach.

The alginate, hydroxycitric acid, and garcinol of the present invention can work synergistically to improve the appetite suppression; and, thereby, are believed to improve body mass index, and blood glucose, insulin, and triglyceride levels. The alginate, hydroxycitric acid, and piperine of the present invention can also work synergistically to improve the appetite suppression; and, thereby, are believed to improve body mass index, and blood glucose, insulin, and triglyceride levels.

In another embodiment, the appetite suppressant composition can include about 0.01% to 10% (w/v) alginate, about 0.0002% to 1% (w/v) piperine, and about 0.0001% to 1% (w/v) hydroxycitric acid.

The following example of one embodiment of the present invention provides an appetite suppressant composition in about 500 mL of water and demonstrates the scope of the present invention. The appetite suppressant composition includes:

about 0.5 g alginate;

about 5 mg piperine; and

about 900 mg hydroxycitric acid.

The following example of one embodiment of the present invention provides an appetite suppressant composition in about 500 mL of water and demonstrates the scope of the present invention. The appetite suppressant composition includes:

about 0.1% (w/v) alginate;

about 0.001% (w/v) piperine; and

about 0.18% (w/v) hydroxycitric acid.

Another example of one embodiment of the present invention provides an appetite suppressant composition in about 100 mL of water and demonstrates the scope of the present invention. The appetite suppressant composition includes:

about 0.5% (w/v) alginate;

about 0.005% (w/v) piperine; and

about 0.9% (w/v) hydroxycitric acid.

A further example of one embodiment of the present invention provides an appetite suppressant composition in water and demonstrates the scope of the present invention. The appetite suppressant composition includes:

about 0.5 g alginate;

about 5 mg piperine; and

about 900 mg hydroxycitric acid.

In another embodiment, the appetite suppressant composition can include about 0.01% to 10% (w/v) alginate, and about 0.0001% to 1% (w/v) of the composition including hydroxycitric acid and garcinol. In another embodiment, the appetite suppressant composition can include about 0.01% to 10% (w/v) alginate, and about 0.0001% to 1% (w/v) GARCITRIN®.

The following example of one embodiment of the present invention provides an appetite suppressant composition in about 500 mL of water and demonstrates the scope of the present invention. The appetite suppressant composition includes:

about 0.5 g alginate; and

about 900 mg of the composition including hydroxycitric acid and garcinol.

The following example of one embodiment of the present invention provides an appetite suppressant composition in about 500 mL of water and demonstrates the scope of the present invention. The appetite suppressant composition includes:

about 0.1% (w/v) alginate; and

about 0.18% (w/v) of the composition including hydroxycitric acid and garcinol.

Another example of one embodiment of the present invention provides an appetite suppressant composition in about 100 mL of water and demonstrates the scope of the present invention. The appetite suppressant composition includes:

about 0.5% (w/v) alginate; and

about 0.9% (w/v) of the composition including hydroxycitric acid and garcinol.

A further example of one embodiment of the present invention provides an appetite suppressant composition in water and demonstrates the scope of the present invention. The appetite suppressant composition includes:

about 0.5 g alginate; and

about 900 mg of the composition including hydroxycitric acid and garcinol.

In another embodiment, the alginate can have a high concentration of glucuronic acid residues to provide improved alginate gel formation. In a further embodiment, the alginate can include about 50% or more glucuronic acid. In a further embodiment, the alginate can include about 66% or more glucuronic acid.

In another embodiment, the appetite suppressant composition can also include calcium carbonate and/or calcium lactate to provide improved alginate gel formation. The alginate forms the best gel in the presence of divalent cations, such as calcium ions. Suitably the calcium carbonate and/or calcium lactate can be encapsulated wherein the calcium carbonate and/or calcium lactate does not release until the appetite suppressant composition reaches the stomach and the alginate gel is formed. The calcium carbonate and/or calcium lactate can release when the stomach is reached and the calcium carbonate and/or calcium lactate contacts gastric juices.

In another embodiment of the invention, the appetite suppressant composition can also include sodium and/or potassium bicarbonate to maintain the alginate gel in the stomach for as long as possible. Sodium bicarbonate and/or potassium bicarbonate can react with and/or release carbon dioxide upon mixing with stomach acids and the carbon dioxide can then be trapped within the alginate gel during formation. The alginate gel floats on the stomach contents thereby delaying gastric emptying which extends satiety and prevents stomach acids from refluxing and/or entering the esophagus.

In one embodiment of the invention, the appetite suppressant composition can also include a chelated source of calcium to assist the alginate in gel formation. At a neutral pH, the chelated source of calcium is bound to the chelating agent. Upon ingestion, the pH of the appetite suppressant composition is lowered by the gastric acid and the chelated source of calcium is released by the chelating agent and the released calcium can help in the alginate gel formation. Suitably, the chelating agent can be alpha lactalbumin (preferably enriched with calcium).

In another embodiment, the appetite suppressant composition can include about 0.01% to 10% (w/v) alginate, about 0.0001% to 1% (w/v) hydroxycitric acid, about 0.0002% to 1% (w/v) piperine, about 0.005% to 0.16% (w/v) sodium and/or potassium bicarbonate, about 0.008% to 0.08% (w/v) calcium, and about 0.02% to 0.4% (w/v) alpha lactalbumin.

In a further embodiment, the appetite suppressant composition can include about 0.1% (w/v) alginate, about 0.001% (w/v) piperine, about 0.18% (w/v) hydroxycitric acid, about 0.12% (w/v) sodium and/or potassium bicarbonate, about 0.032% (w/v) calcium, and about 0.2% (w/v) alpha lactalbumin, in about 500 ml of water.

In a further embodiment, the appetite suppressant composition can include about 0.5 g alginate, about 5 mg piperine, about 900 mg hydroxycitric acid, about 600 mg sodium and/or potassium bicarbonate, about 160 mg calcium, and about 1 g alpha lactalbumin, in about 500 ml of water.

In a further embodiment, the appetite suppressant composition can include about 0.5 g alginate, about 5 mg piperine, about 900 mg hydroxycitric acid, about 600 mg sodium and/or potassium bicarbonate, about 160 mg calcium, and about 1 g alpha lactalbumin, in water.

In another embodiment, the appetite suppressant composition can include about 0.01% to 10% (w/v) alginate, about 0.0001% to 1% (w/v) of the composition including hydroxycitric acid and garcinol, about 0.005% to 0.16% (w/v) sodium and/or potassium bicarbonate, about 0.008% to 0.08% (w/v) calcium, and about 0.02% to 0.4% (w/v) alpha lactalbumin.

In a further embodiment, the appetite suppressant composition can include about 0.1% (w/v) alginate, about 0.18% (w/v) of the composition including hydroxycitric acid and garcinol, about 0.12% (w/v) sodium and/or potassium bicarbonate, about 0.032% (w/v) calcium, and about 0.2% (w/v) alpha lactalbumin, in about 500 ml of water.

In a further embodiment, the appetite suppressant composition can include about 0.5 g alginate, about 900 mg of the composition including hydroxycitric acid and garcinol, about 600 mg sodium and/or potassium bicarbonate, about 160 mg calcium, and about 1 g alpha lactalbumin, in about 500 ml of water.

In a further embodiment, the appetite suppressant composition can include about 0.5 g alginate, about 900 mg of the composition including hydroxycitric acid and garcinol, about 600 mg sodium and/or potassium bicarbonate, about 160 mg calcium, and about 1 g alpha lactalbumin, in water.

In another embodiment, the appetite suppressant composition can include a preservative. The preservative can include parabens and can further include methyl and propyl parabens. Parabens are common preservatives in the cosmetic, food and beverage, and pharmaceutical industries due to their anti-microbial effects. In the U.S., parabens are GRAS approved and allowed at a maximum concentration of 0.1%. In a further embodiment, the appetite suppressant composition can include methyl and propyl parabens in a 3:1 or 2:1 ratio. In a further embodiment, the appetite suppressant composition can include 0.375 g of methyl paraben and 0.125 g of propyl paraben.

In another embodiment, the appetite suppressant composition can be further sterilized and have an extended shelf-life through an ultra-high temperature process. The ultra-high temperature process includes heating the appetite suppressant composition for about 2-3 seconds at a temperature of at least approximately 275 degrees Fahrenheit. The ultra-high temperature process aids in killing microbes present in the appetite suppressant composition prior to mixing in the spring water.

One form of the invention also relates to a method of suppressing appetite. One form of the method of suppressing appetite includes administering the appetite suppressant composition described above. The method also includes administering an appetite suppressant composition comprising alginate, hydroxycitric acid, and garcinol, and the alginate forming a gel in the stomach of the human creating an immediate and sustained feeling of satiety. The method also includes administering an appetite suppressant composition comprising alginate, hydroxycitric acid, and piperine, and the alginate forming a gel in the stomach of the human creating an immediate and sustained feeling of satiety. The method can further include the hydroxycitric acid blocking the conversion of carbohydrates into fat and the alginate binding to cholesterol reducing total cholesterol, glucose, insulin and triglyceride levels. The method can also further include the GLP-1 and ghrelin hormones reducing hunger and creating a feeling of satiety. In one embodiment, the method of the invention can include administering an appetite suppressant composition comprising alginate, hydroxycitric acid, piperine, and encapsulated calcium carbonate and/or calcium lactate, and the encapsulated calcium carbonate and/or calcium lactate forming a superior gel in the stomach of the human creating an immediate and sustained feeling of satiety. In another embodiment, the method of the invention can include administering to a human an appetite suppressant composition comprising alginate, hydroxycitric acid, piperine, and sodium and/or potassium bicarbonate, and the sodium and/or potassium bicarbonate creating a longer lasting alginate gel in the stomach.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. An appetite suppressant composition, comprising:
alginate;
hydroxycitric acid; and
a hydroxycitric acid bioavailability increasing component.

2. The composition of claim 1 wherein the hydroxycitric acid bioavailability increasing component is garcinol.

3. The composition of claim 2 wherein the hydroxycitric acid and the garcinol are a single ingredient.

4. The composition of claim 1 wherein the composition is in a beverage form.

5. The composition of claim 1 wherein the hydroxycitric acid comprises sodium hydroxycitric acid, potassium hydroxycitric acid, calcium hydroxycitric acid or a combination thereof.

6. The composition of claim 1 further comprising encapsulated calcium.

7. The composition of claim 6 wherein the encapsulated calcium comprises calcium carbonate, calcium lactate or a combination thereof.

8. The composition of claim 1 further comprising sodium bicarbonate, potassium bicarbonate or a combination thereof.

9. The composition of claim 1 further comprising a chelated source of calcium.

10. The composition of claim 9 wherein the chelated source of calcium comprises calcium and alpha lactalbumin as a chelating agent.

11. The composition of claim 1 wherein the alginate comprises about 50% or more glucuronic acid.

12. The composition of claim 11 further comprising about 66% or more glucuronic acid.

13. The composition of claim 3 wherein the alginate is about 0.01% to 10% (w/v), and the single ingredient of hydroxycitric acid and garcinol is about 0.0001% to 1% (w/v), in water.

14. The composition of claim 3 wherein the alginate is about 0.5 g to 50 g and the single ingredient of hydroxycitric acid and garcinol is about 50 mg to 5 g, in water.

15. The composition of claim 3 wherein the alginate is about 0.5 g and the single ingredient of hydroxycitric acid and garcinol is about 900 mg, in water.

16. The composition of claim 1 wherein the hydroxycitric acid bioavailability increasing component is piperine.

17. The composition of claim 16 wherein the alginate is about 0.01% to 10% (w/v), the hydroxycitric acid is about 0.0001% to 1% (w/v), and the piperine is about 0.0002% to 1% (w/v), in water.

18. The composition of claim 16 wherein the alginate is about 0.5 g to 50 g, the hydroxycitric acid is about 50 mg to 5 g, and the piperine is about 1 mg to 5 g, in water.

19. The composition of claim 16 wherein the alginate is about 0.5 g, the hydroxycitric acid is about 900 mg, and the piperine is about 5 mg, in water.

20. The composition of claim 16 wherein the piperine is in the form of a piperine/dextrin complex.

21. The composition of claim 1 further comprising a flavoring component, a sweetening component and combinations thereof.

22. An appetite suppressant composition, comprising:
at least about 0.1% (w/v) alginate; and
at least about 0.18% (w/v) a single ingredient of hydroxycitric acid and garcinol,
in 500 mL of water.

23. The composition of claim 22 further comprising:
at least about 0.12% (w/v) sodium bicarbonate, potassium bicarbonate or a combination thereof;
at least about 0.032% (w/v) calcium; and
at least about 0.2% (w/v) alpha lactalbumin.

24. The composition of claim 22 further comprising:
about 1 to 3 drops of fruit concentrate.

25. An appetite suppressant composition, comprising:
at least about 0.1% (w/v) alginate;
at least about 0.001% (w/v) piperine; and
at least about 0.18% (w/v) hydroxycitric acid,
in 500 mL of water.
